(19) United States
(12) Patent Application Publication
(10) Pub. No.: US 2011/0270411 A1
(43) Pub. Date: Nov. 3, 2011

(54) NERVE GRAFT PREPARED BY ELECTROSTATIC SPINNING, THE PREPARING METHOD AND THE SPECIAL APPARATUS USED THEREFOR

(75) Inventors: Yumin Yang, Jiangsu (CN); Xiaosong Gu, Jiangsu (CN); Fei Ding, Jiangsu (CN); Shanqing Xu, Jiangsu (CN); Xiaoli Yan, Jiangsu (CN)

(73) Assignee: NANTONG UNIVERSITY

(21) Appl. No.: 12/998,651

(22) PCT Filed: Mar. 31, 2010
(86) PCT No.: PCT/CN2010/071471

§ 371 Date: May 13, 2011

(30) Foreign Application Priority Data
Sep. 2, 2009 (CN) 200910034583.9

(51) Int. Cl. A61F 2/02; B29C 47/00; D01F 9/00

(52) U.S. Cl. 623/23.72; 264/484; 425/174.8 E

(57) ABSTRACT

The present invention discloses an artificial nerve graft prepared by electrostatic spinning, the preparing method and a special apparatus used therefor. Said artificial nerve graft is in the shape of a tube composed of nano-fiber that is prepared by electrostatic spinning of a polymer. The materials used in the present invention are bio-degradable materials and of desirable biocompatibility with human body. The product of the present invention is free of exogenous toxic substances or substances having side effects. Furthermore, the tube wall is of a 3-dimensional structure having micropores contained therein thereby providing a path for supplying nutritions required for the growth of nerve cells. Another advantage of the present invention is that necessary induction and space are provided for the growth of the nerve cells.

### TECHNICAL FIELD OF THE INVENTION

The present invention relates to an artificial nerve graft used for bridging the nerves around a damage, and improving the regeneration of the same. The present invention also provides a method for preparing the artificial nerve graft.

### TECHNICAL BACKGROUND OF THE INVENTION

The development and modernization of the society as well as increasing pace of life is accompanied by more and more traffic accidents, work injuries and unexpected sport accidents. Meanwhile, frequent regional conflicts, violence and natural disasters such as earthquakes would also cause casualties including nerve damages to humans. Clinically, when a medium or long distance nerve damage can not be repaired by end-to-end stitching, a nerve graft would be required to bridge the damaged nerves. Appropriate nerve grafts have been searched and studied for more than 100 years. However, except for autologous nerves being used as the preferred grafts for bridging the damaged nerves, ideal replacements of autologous nerves that can be widely clinically used have not been found. Even the clinical use of autologous nerves for bridging the damaged nerves has limitations due to the limited source of the autologous nerves, hard-to-match of the structure and size as well as lacking innervations in the nerve supply region for a long period of time.

With the development of tissue engineering, a new way for preparing the replacements for autologous nerve grafts emerged. Currently, two ways for preparing artificial nerve grafts are known in the art. One way is using the original tube of the nerve by removing allogeneic cells from the tube of the allogenic nerves. The other way is preparing artificial nerve grafts by pouring a solution into an appropriate mould, such as the artificial chitosan nerve grafts and the artificial silk fibroin nerve grafts we have prepared.

Electrostatic spinning is one of the important methods for preparing nano-fiber. The essence of the technology is: under the effect of a high voltage electrostatic of thousands to tens of thousands volts, the electric field force surmounts the surface tension of the polymer solution thereby the polymer solution would spray to form a stable electric stream of spray fluid. Finally, the solvent would vaporize and the electric stream would be collected by a collector under the effect of the electric field and thus obtain the product, i.e. a fiber web or a membrane in the form similar to non-woven fabrics, which is composed of nano-fibers.

The product obtained by electrostatic spinning is of a very big specific surface area and holes of different sizes. The nano-fiber web structure is similar to the structure of extracellular matrix and is beneficial to the adhesion of cells and thus improves the regeneration of the tissues.

### SUMMARY OF THE INVENTION

The purpose of the present invention is to provide an artificial nerve graft, the preparing method and a special apparatus used therefor. The artificial nerve graft of the present invention is beneficial to the adhesion of cells and can improve the regeneration of the tissues.

The technical solution of the present invention is:

An artificial nerve graft prepared by electrostatic spinning wherein said artificial nerve graft is in the shape of a tube composed of nano-fibers that is prepared by electrostatic spinning of a polymer.

Said polymer can be selected from the group consisting of silk protein, chitosan, polyglycolic acid, polycaprolactone, collagen, polylactic acid, gelatin, or any combination thereof.

A method for preparing the artificial nerve graft obtained by electrostatic spinning comprises the steps of: dissolving a polymer in a solvent to obtain a solution; spraying the solution by an electrostatic spinning solution sprayer to a collector roller moving back and forth while rotating to form nano-meter fibers and collecting the same on the collector roller to form a tube; removing the tube, treating the tube to obtain the tube-shape artificial nerve graft.

A special apparatus for preparing the artificial nerve graft obtained by electrostatic spinning comprises a micro measuring pump, a solution sprayer, a collector roller and a high voltage static generator; said micro measuring pump is connected to said solution sprayer; said collector roller moving back and forth while rotating is arranged in the spraying direction of said solution sprayer; said high voltage static generator is used for providing a high voltage static.

The special device for preparing the artificial nerve graft by electrostatic spinning can further comprise a control unit to control said micro measuring pump, said solution sprayer, and said collector roller.

In the process of preparing the artificial nerve graft described as above, or after the preparation of the artificial nerve graft, medicative cytokines, such as a nerve growth factor (NGF), neurotrophic factor-3 (NT-3), a brain-derived neurotrophic factor (BDNF), a glial cell line-derived neurotrophic factor (GDNF), or any combination thereof can be added.

The artificial nerve graft as prepared can also be added with medicative seed cells. Said seed cells can be selected from the group consisting of mesenchymal stem cells, neural stem cells, schwann cells, olfactory ensheathing cells, embryonic stem cells, or any combination thereof.

The materials used in the present invention are bio-degradable materials and of desirable biocompatibility with human body. The product of the present invention is free of exogenous toxic substances or substances having side effects. Furthermore, the tube wall is of a 3-dimensional structure having micropores contained therein thereby providing a path for supplying nutritions required for the growth of nerve cells. Another advantage of the present invention is that necessary induction and space are provided for the growth of the nerve cells.

When cultured along with nerve tissue cells in vitro, it was found that the product of the present invention was of good histocompatibility based on the results of morphologic observation, determination of enzyme metabolism activity as well as determination of expression of nerve growth factor. The product of the present invention was also applied to repair a 1 cm sciatic nerve damage in the body of a rat and the result indicated that said product was beneficial to the growth of the nerves. It was also found that the functions of the damaged nerves were revived. Meanwhile, the product of the present invention was of good biocompatibility.

Since the electrostatic spinning product of the present invention has a relatively high specific area and micropores of different size, and its web structure formed by nanometer fibers is similar to the extracellular matrix structure, it is very beneficial to the adhesion of cells thereby improving the regeneration of tissues. Therefore, the artificial nerve graft of the present invention exhibits not only good biocompatibility and biodegradability, but also desirable mechanical properties. Furthermore, the artificial nerve graft can also be combined with therapeutic cell factors, medicaments and seed cells.

### BRIEF DESCRIPTION OF THE DRAWINGS

FIG. 1 is a schematic diagram of the special apparatus for preparing the artificial nerve graft of the present invention.

### DETAILED DESCRIPTION OF THE INVENTION

The present invention will be further described by referring to the accompanying drawings and examples.

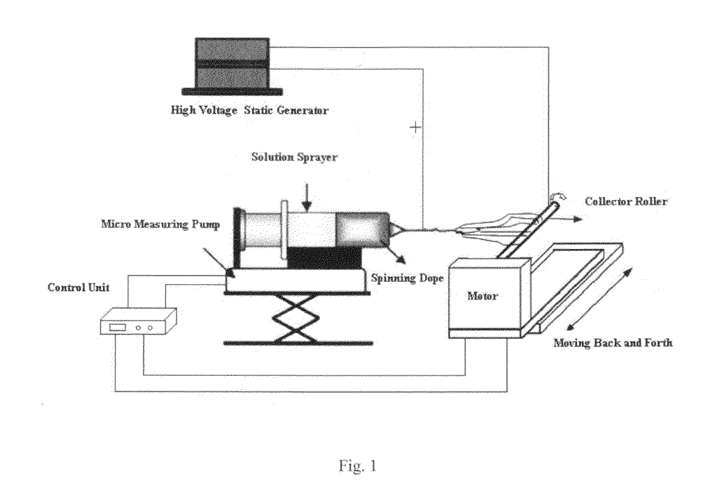

As shown in FIG. 1, the special apparatus for preparing the artificial nerve graft by electrostatic spinning of the present invention comprises a micro measuring pump 1, a solution sprayer 2, a collector roller 3 and a high voltage static generator 5; said micro measuring pump 1 is connected to said solution sprayer 2; said collector roller 3 moving back and forth while rotating is arranged in the spraying direction of said solution sprayer 2; said high voltage static generator 5 is used to provide a high voltage static.

The special apparatus for preparing the artificial nerve graft by electrostatic spinning can further comprise a control unit 4 to control said micro measuring pump 1, said solution sprayer 2, and said collector roller 3.

### Example 1

The weak caustic solution (0.1-10% sodium carbonate solution or 0.1-10% potassium carbonate solution) containing natural silk is first heated to 50-100° C. and the fibers after the treatment are cleaned with distilled water to obtain silk protein fibers; at 25-80° C. (for example, 25° C., 50° C. or 80° C.), the natural silk protein fibers prepared as above are dissolved in a mixture of calcium chloride, ethanol and water having a molar ratio of 1:2:8 (calcium chloride:ethanol:water); after 0.5-6 hours (for example, 0.5, 3 or 6 hours), the dissolved mixture is then put into a cellulose membrane bag and dialysized by distilled water.

The dialysized silk solution is then poured into a plane mould. After dried, a silk protein membrane is obtained. The silk protein membrane is dissolved using formic acid to obtain a spinning dope having a concentration of 13% by weight. Said spinning dope is shaped by using the special apparatus for preparing the artificial nerve graft by electrostatic spinning, which is described as above. The spinning dope is first measured by the micro measuring pump and introduced to the solution sprayer and is sprayed therefrom onto the collector roller moving back and forth while rotating to form nano-fibers that are collected on the collector roller to obtain a tube. In the process of shaping, the voltage of the high voltage static generator is 20 KV; the solution spraying rate is 0.3 ml/h; the distance from the end of the solution sprayer to the collector roller is 7-11 cm; the average speed of moving back and forth is 1.5 m/h, and the rotating speed of the collector roller is 150 r/min. The silk protein tube that is preliminarily shaped is put in an ethanol solution for post-treatment and then is cleaned by distilled water to obtain the nano-fiber silk protein artificial nerve graft.

### Example 2

Chitosan is dissolved in an acetic acid (or phosphoric acid, citric acid, lactic acid) having a concentration of 2-15% (for example 2%, 8% or 15%) and a predetermined concentration (for example 5%, 25%, or 50%) of collagen solution is added therein to prepare a spinning dope having a concentration of 10% by weight. Said spinning dope is shaped by using the special apparatus for preparing the artificial nerve graft by electrostatic spinning, which is described as above. The spinning dope is first measured by the micro measuring pump and introduced to the solution sprayer and sprayed therefrom onto the collector roller moving back and forth while rotating to form nano-fibers that are collected on the collector roller to obtain a tube. In the process of shaping, the voltage of the high voltage static generator is 25 KV; the solution spraying rate is 0.2 ml/h; the distance from the end of the solution sprayer to the collector roller is 8-11 cm; the average speed of moving back and forth is 2 m/h, and the rotating speed of the collector roller is 90 r/min. The silk protein tube that is preliminarily shaped is first cleaned in a 1 mol/L sodium hydroxide solution and then cleaned in a 50 mmol/L phosphate buffer and finally cleaned in distilled water to obtain a nano-fiber chitosan/collagen artificial nerve graft.

### Example 3

Polyglycolic acid (PGA), polylactic acid (PLA), or a copolymer of glycolic acid and lactic acid (PLGA, 50/50) is dissolved in a chloroform to prepare a spinning dope having a concentration of 10-20% by weight. Said spinning dope is shaped by using the special apparatus for preparing the artificial nerve graft by electrostatic spinning, which is described as above. The spinning dope is first measured by the micro measuring pump and introduced to the solution sprayer and sprayed therefrom onto the collector roller moving back and forth while rotating to form nano-fibers that are collected on the collector roller to obtain a tube. In the process of shaping, the voltage of the high voltage static generator is 20-30 KV; the solution spraying rate is 0.2 ml/h; the distance from the end of the solution sprayer to the collector roller is 7-11 cm; the average speed of moving back and forth is 2 m/h, and the rotating speed of the collector roller is 70-130 r/min. The silk protein tube that is preliminarily shaped is put in an ethanol solution for post-treatment and then is cleaned by distilled water to obtain a nano-fiber polyglycolic acid, polylactic acid or copolymer of glycolic acid and lactic acid artificial nerve graft.

What is claimed is:

1. An artificial nerve graft prepared by electrostatic spinning wherein said artificial nerve graft is in the shape of a tube composed of nano-fiber that is prepared by electrostatic spinning of a polymer.

2. The artificial nerve graft prepared by electrostatic spinning according to claim 1, wherein said polymer is selected from the group consisting of silk protein, chitosan, polyglycolic acid, polycaprolactone, collagen, polylactic acid, gelatin, or any combination thereof.

3. A method for preparing the artificial nerve graft by electrostatic spinning comprises the steps of:
dissolving a polymer in a solvent to obtain a spinning dope;
spraying the spinning dope by an electrostatic spinning solution sprayer onto a collector roller moving back and forth while rotating to form nano-meter fiber and collecting the same on the collector roller to form a tube;
removing the tube, treating the tube to obtain a tube-shape artificial nerve graft.

4. The method for preparing the artificial nerve graft by electrostatic spinning according to claim 3, wherein a 0.1-10% sodium carbonate solution or 0.1-10% potassium carbonate solution containing natural silk is first heated to 50-100° C. and the fibers after the treatment are cleaned with distilled water to obtain silk protein fibers; at 25-80, the silk protein fibers are then dissolved in a mixture of calcium chloride, ethanol and water having a molar ratio of 1:2:8; after 0.5-6 hours, the dissolved mixture is put into a cellulose membrane bag and dialysized by distilled water; the dialysized silk solution is poured into a plane mould; after dried, a silk protein membrane is obtained; the silk protein membrane is dissolved by formic acid to obtain a spinning dope having a concentration of 13% by weight; said spinning dope is shaped by using the special apparatus for preparing the artificial nerve graft by electrostatic spinning and wherein the spinning dope is first measured by the micro measuring pump and introduced to the solution sprayer and sprayed therefrom onto the collector roller moving back and forth while rotating to form nano-fibers that are collected on the collector roller to obtain a tube; the voltage of the high voltage static generator is 20 KV; the solution spraying rate is 0.3 ml/h; the distance from the end of the solution sprayer to the collector roller is 7-11 cm; the average speed of moving back and forth is 1.5 m/h, and the rotating speed of the collector roller is 150 r/min; the silk protein tube that is preliminarily shaped is put in a ethanol solution for post-treatment and then is cleaned by distilled water to obtain the nano-fiber silk protein artificial nerve graft.

5. The method for preparing the artificial nerve graft by electrostatic spinning according to claim 3, wherein chitosan is dissolved in a weak acid solution having a concentration of 2-15%, said weak acid solution is acetic acid, phosphoric acid, citric acid, or lactic acid; and 5-25% of collagen solution is added therein to prepare a spinning dope having a concentration of 10% by weight; said spinning dope is shaped by electrostatic spinning wherein the voltage is 25 KV; the solution spraying rate is 0.2 ml/h; the distance from the end of the solution sprayer to the collector roller is 8-11 cm; the average speed of moving back and forth is 2 m/h, and the rotating speed of the collector roller is 90 r/min; and the silk protein tube that is preliminarily shaped is first cleaned in a 1 mol/L sodium hydroxide solution and then cleaned in a 50 mmol/L phosphate buffer and finally cleaned in distilled water to obtain a nano-fiber chitosan/collagen artificial nerve graft.

6. The method for preparing the artificial nerve graft by electrostatic spinning according to claim 3, wherein polyglycolic acid, polylactic acid, or a copolymer of glycolic acid and lactic acid (PLGA, 50/50) is dissolved in a chloroform to prepare a spinning dope having a concentration of 10-20% by weight; said spinning dope is shaped by electrostatic spinning wherein the voltage is 20-30 KV; the solution spraying rate is 0.2 ml/h; the distance from the end of the solution sprayer to the collector roller is 7-11 cm; the average speed of moving back and forth is 2 m/h, and the rotating speed of the collector roller is 70-130 r/min; the silk protein tube that is preliminarily shaped is put in a ethanol solution for post-treatment and then is cleaned by distilled water to obtain a nano-fiber polyglycolic acid, polylactic acid or copolymer of glycolic acid and lactic acid artificial nerve graft.

7. A special apparatus for preparing the artificial nerve graft by electrostatic spinning comprises a micro measuring pump, a solution sprayer, a collector roller and a high voltage static generator; said micro measuring pump is connected to said solution sprayer; said collector roller moving back and forth while rotating is arranged in the spraying direction of said solution sprayer; said high voltage static generator is used to provide a high voltage static, and wherein said special apparatus for preparing the artificial nerve graft by electrostatic spinning further comprises a control unit to control said micro measuring pump, said solution sprayer, and said collector roller.
